(19) United States
(12) Patent Application Publication
(10) Pub. No.: US 2010/0179089 A1
(43) Pub. Date: Jul. 15, 2010

(54) Compositions and Methods to Manage the Inflammatory Basis of Chronic Disease Conditions and Maintain an Optimal Immune Response in Elderly

(76) Inventors: Nicolaas E.P. Deutz, Little Rock, AR (US); John P. Troup, Plymouth, MN (US); Robert R. Wolfe, Little Rock, AR (US)

Correspondence Address: LEWIS, RICE & FINGERSH, LC;ATTN: BOX IP DEPT., 600 Washington Ave., Suite 2500, ST LOUIS, MO, 63101, US

(21) Appl. No.: 12/352,899

(22) Filed: Jan. 13, 2009

(51) Int. Cl. A61K 38/02; A61K 31/20; A61K 31/70; A61P 29/00

(52) U.S. Cl. 514/2; 514/560; 514/23

(57) ABSTRACT

Compositions and methods for managing inflammation in the elderly by delivering a selection of amino acids, including arginine and/or citrulline, in a synergistic ratio with omega-3 fatty acids and carbohydrates with a low glycemic index.

### BACKGROUND

1. Field of the Invention

The present invention relates to nutritional compositions for managing inflammation in the elderly from any source. In particular, to nutritional compositions comprising essential amino acids, including arginine and/or citrulline, and omega-3 fatty acids.

2. Description of the Related Art

Inflammation is believed to be the underlying problem of compromised health conditions in an aging population, and is an important health care problem around the world. Inflammation refers to symptoms including redness, swelling, pain, stiffness, and loss of joint function. More general symptoms such as fever, chills, fatigue, loss of appetite, and muscle stiffness may also be present. Inflammation of particular organ systems may lead to more serious symptoms, such as shortness of breath, asthma, high blood pressure, kidney failure, or cramping. The symptoms are often caused by a misdirected immune response, often mediated by cytokines or inflammation mediators, which are molecules involved in the immune response that trigger or contribute to inflammation. Disorders that compromise health status with underlying inflammation include the chronic states of diabetes, Chronic Obstructive Pulmonary Disease (COPD), congestive heart failure, sarcopenia, and cachexia.

Inflammation is believed to be particularly prevalent in the elderly, including but not limited to individuals over sixty (60), due to the aging of the mitochondria. As shown in FIG. 1, mitochondria produce reactive oxygen species (ROS) as a byproduct of oxidative metabolism. Oxidative stress can activate NFkB and promote the expression of proinflammatory cytokines such as IL-1b, IL-6, TNF-a and b. It is desirable to reduce and overcome the production of these cytokines in order to reduce inflammation and decrease risk of compromised health status, in a manner believed to be appropriate for the elderly.

Hyperinflammed states in the elderly result in a compromised immune cascade and response that in turn may result in co-morbidities and a reduction in the ability of the individual to remain healthy. Inflammation may also contribute to the early stages of cognitive decline and, as observed in an elderly hospitalized patient group in the acute care state, leads to greater infectious complications and compromises patient outcomes. The increased presence of inflammatory mediators during the aging process puts the aged body in a state of constant, low-grade inflammation, compromising health and threatening life.

Chronic inflammation causes numerous adverse side effects. Generally, elevated serum levels of proinflammatory mediator's are associated with poor health outcomes including greater disability, frailty, and mortality in healthy older adults. Chronic inflammation is also believed to accelerate muscle breakdown when the supply of protein and energy from the diet is insufficient to meet the body's demand. Consequently, the peripheral muscles shrink and the elderly person experiences unintentional loss of weight and muscle strength. Elderly persons with chronic inflammation, low muscle reserves, and unintentional weight loss are bound for poor health outcomes.

Further, individuals with chronic inflammation are predisposed to developing chronic disease and co-morbidities and are at higher risk of infection. For example, persons with greater inflammation are more likely to have diabetes and impaired cognitive function; those with elevated C-reactive protein (“CRP”, an inflammation mediator) are at greater risk of cardiovascular complications; and those with increased IL-6 production (another inflammation mediator) are more likely to suffer loss of muscle mass, strength, and function. Observation of hospitalized elderly patients has revealed these patients are also in a hyperinflammed state which in turn puts them at greater risk of infection, infectious complications, and prolonged recovery and delayed release from the hospital. Increasing evidence suggests that chronic elevation in inflammatory mediator's negatively impacts endocrine, skeletal muscle, and clotting systems as well as glucose metabolism.

Chronic inflammation is also believed to be responsible for vascular complications, known to be the leading cause of morbidity and mortality in people with diabetes, for example IL-6 contributes to atherosclerosis by initiating the induction of adhesion molecules, stimulating smooth muscle cell proliferation and increasing endothelial permeability. Elevated IL-6 is a major stimulus for production of most acute phase proteins including CRP, which is believed to be an excellent predictor of cardiovascular disease.

Because inflammation has so many widespread and severe comorbidities, it is desirable to address inflammation. It is further desirable to do so noninvasively and in a manner that encourages patient compliance, particularly by elderly patients for whom invasive treatment may be particularly inappropriate. It is further desirable that the treatment address multiple comorbidities of inflammation in the elderly, including: poor diet, a depressed immune system, and muscle wasting and diminished muscle synthesis.

The nutritional practices and habits of the elderly often fail the recommended dietary guidelines, such that nutrient deficiencies and malnutrition often contribute to physiological changes and a compromised immune response system. These changes include an observed shift in body mass which may further accentuate underlying inflammation. An elevated fat mass/fat free mass ratio that occurs about after age 45 results in an increased production of inflammatory markers. In this chronic inflammatory state, enhanced levels of CRP, TNFa, and IL6 (inflammation mediators) are commonly observed and are believed to inhibit muscle protein synthesis.

The elderly are also believed to suffer from a significant decrease in both total calories consumed (about a 30 to 40% decrease) and a decrease in intake of critical macronutrients, in particular protein and fat calories. More specifically, the amount of protein and fat consumed may decrease by up to about 40% such that minimum energy requirements of the elderly can not be met. Under conditions of inadequate nutrient availability, the body will catabolize peripheral muscle as a source of protein to provide the body with amino acids and energy, resulting in muscle wasting.

Muscle wasting has been shown to be related to an increase in the inflammation mediator TNF. Through a separate mitochondrial mechanism, TNFa acts to increase reactive oxygen species (ROS), or free radicals, that ultimately result in more robust breakdown of muscle tissue. Segments of the elderly population that are immobile or bed-ridden due to illness may suffer the greatest from muscle lost through this mechanism. Muscle protein synthesis is believed to be 30% lower in older adults, making the ability to regenerate skeletal muscle following injury or overload difficult with age. Thus, it is more difficult for elderly persons to reverse the effects of protein-energy malnutrition and regain muscle lost due to the stress of acute or chronic conditions. With protein undernutrition, availability of the essential amino acids is limited and adequate protein synthesis rates to maintain body weight is not possible. Recent studies have suggested that that when macronutrient deficiencies in protein and fat occur, protein synthetic rates are compromised.

Because a poor, low-protein and high-fat diet is believed to facilitate inflammation, and is prevalent in the elderly population, it is desirable to address inflammation through improving the diet, which also has many other beneficial effects such as improving protein synthesis. Other contributors to the protein synthesis pathway, such as ribose and key amino acid precursors which participate in rate-limiting steps in protein synthesis, may also be desirable to include in a composition for improving inflammation.

Another common problem in the elderly, infection, also causes inflammation. Aging is associated with an impaired immune response and function, A key contributor to the increased incidence of infection with age is the aging-associated dysregulation of immune function. Also known as immune senescence, immune system dysregulation with advancing age is believed to occur primarily due to diminished T-cell-mediated functions and a decrease in the number of naïve T cells. Overall, the elderly are less able to respond to new antigens or latent infections Aging is also associated with an increased prevalence of auto-antibodies that increase the incidence of degenerative and autoimmune diseases, such as rheumatoid arthritis.

The amino acid arginine is believed to contribute to normal T cell proliferation and function. A decrease in arginine is believed to cause significant alterations in T cell structure, including a decrease in CD3 receptors and a decrease in the ζ-chain subunit of the T cell receptor. However, arginine deficiency is very prevalent in the elderly, due to reduced food intake and other causes. In the myeloid cells of the elderly, increased expression of the protein Arginase I is observed; Arginase I in myeloid cells can deplete arginine. Through arginine depletion, Arginase I appears to play a major role in T cell dysfunction in aging as well as after trauma or surgery and in preventing the production of nitric oxide in response to infection. It is believed that Arginase I expression in myeloid cells is induced by prostaglandin E2 (“PGE2”). Arginine synthesis and degradation pathways are shown in FIG. 6.

In addition, the elderly have a reduced capability to produce arginine endogenously. Recently, it was observed that de novo arginine production is substantially reduced during sepsis in the elderly, while the young do not experience such a decrease (FIG. 2). Aging is sometimes characterized by a decline in renal glomerular filtration rate (GFR) that has been linked to reduced renal arginine de novo production and synthesis of nitric oxide, important to normal functioning of the immune system.

In addition to contributing to a compromised immune system, a relative deficiency of arginine may play a role in sarcopenia, or diminished muscle synthesis, in the elderly. Reduced arginine concentrations in blood and/or reduced arginine de novo synthetic capacity is believed to enhance muscle protein wasting in mice and pigs. During acute and chronic reduction of arginine levels in different mouse strains and in mice transgenetically modified to have enhanced arginase activity in the gut, a relationship between plasma arginine levels and accelerated whole body protein breakdown was observed. In certain conditions where the intake of arginine is insufficient to meet requirements, supplementation with arginine may lead to anabolism, or muscle synthesis.

For example, the plasma concentration of arginine is believed to be reduced in pigs with a sepsis-like syndrome, indicating an arginine deficiency. Muscle protein synthesis was improved in these pigs by supplementation with arginine. In a second example, arginine supplementation stimulated muscle protein synthesis in rabbits. Muscle protein synthesis was stimulated in anesthetized rabbits when infused with an arginine-rich mixture of amino acids (such as TRAVASOL® (amino acid solution)) that increased plasma arginine four-fold (FIG. 3). Muscle protein breakdown was unchanged when arginine was added to the amino acid solution, meaning that net muscle catabolism was virtually reversed in these post-absorptive rabbits.

It is possible that, in addition to serving as a precursor for protein synthesis, arginine plays a regulatory role in controlling the rate of muscle protein synthesis. It is likely that impaired arginine de novo synthesis of aging can be successfully treated with arginine supplementation. In addition, citrulline, the in vivo precursor of arginine, may provide an alternative route for the generation of arginine.

Given the observed changes in protein intake and observed changes in protein synthesis in aging, it is believed that a hyperinflammed state compromises protein synthesis and further accentuates chronic disease related to arginine deficiency and malnutrition. It is believed that proinflammatory conditions significantly reduce the level of protein synthesis FIG. 4 presents data suggesting that a combination of arginine and other specialized amino acids may stimulate protein synthesis to a greater level than single amino acids alone under conditions of inflammation, yet not at normal or non-inflamed conditions. This data illustrates that for protein synthesis to continue at normal levels of healthy and younger age groups, the level of inflammation must be controlled or overcome.

Because arginine deficiency is believed to contribute to both immune system dysfunction and depressed muscle synthesis, both of which contribute to inflammation, it is desirable to supplement arginine in a treatment for inflammation. It is believed that supplementation with immune enhancing nutrients to protect and improve arginine levels may reduce inflammation, infection rates, and postoperative morbidity; and restore depressed T cell proliferation, increase the production of nitric oxide, and modulate the production of certain inflammatory cytokines or mediators. In order to address diminished muscle synthesis, it may also be desirable to include essential amino acids, i.e. those not made by the human body and in particular arginine, leucine, valine and isoleucine and minimal levels of intake to maximize benefit on maintaining optimal immune function and muscle mass.

Omega-3 fatty acids, including but not limited to alpha-linolenic acid (ALA), eicosapentaenoic (EPA) and docosahexaenoic acids (DHA); omega-6 linoleic acid; and borage oil (also an omega-6 fatty acid) are currently used in different diets with the intention to improve immune dysfunction. ALA, EPA, DHA, and other omega-3 fatty acids and their functional equivalents, which may be derived from fish, flax, eggs, and other sources, may be referred to herein as “omega-3 fatty acids,” Interestingly, the use of arginine or citrulline and EPA or DHA has a strong synergistic action, as supported by FIG. 4 wherein the presence of DHA improves the efficacy of arginine delivery in promoting protein synthesis. Used with omega-3 fatty acids, arginine is believed to be more available to provide a normal response under conditions of inflammation. Dietary intake of omega-3 fatty acids by the elderly may favorably shift the balance in the production of PGE2, via upregulation of Arginase I and downregulation of arginase (as shown in FIG. 6) and, consequently, help avoid arginine deficiency and thereby improve or maintain optimal immune system function. In contrast, prostaglandins from omega-6 fatty acids are believed to significantly increase metabolism of arginine by stimulating its breakdown via stimulation of arginase activity. The products of omega-6 fatty acid metabolism are believed to contribute to arginine deficiency and inflammation in this way. There have been numerous studies showing the benefits of dietary omega-3 fatty acids over omega-6 fatty acids in various physiologic conditions including coronary artery disease, chronic inflammatory conditions such as cancer, and other inflammatory mediated states.

In addition to improving arginine levels, omega-3 fatty acids are also believed to directly mediate plasma levels of inflammatory mediators. Plasma levels of inflammatory mediators rise with a diet rich in omega-6 fatty acids, and fall with a diet rich in omega-3 fatty acids. Additionally, intake of omega-3 fatty acids is also believed to result in improvements in serum IGF-1, decreased IL-6 levels, and an improvement in lean body mass. Further, increased omega-3 fatty acids intake (i.e., docohexanoic acid) is believed to increase the availability and absorption of the antioxidant lutein and overcome the inflammatory effect reflected by increasing production of cytokines or inflammatory mediators. This is supported by FIG. 5, showing changes in lutein and DHA and combinations in serum levels following supplementation.

Omega-3 fatty acids have been used as a co-therapy for immune-related disease and conditions. A randomized controlled trial was recently conducted in adults with active Crohn's disease. Subjects received either a fish-oil enriched diet (enriched in EPA and DHA) having a ratio of omega-6 acids to omega-3 acids of 1.4:1.0; or a similar diet enriched in omega-6 fatty acid that is more like the average diet. Compared to the control group who had elevated IL-6 throughout the study, those who received the fish oil-enriched diet had and maintained lower levels of the proinflammatory cytokine IL-6. While typical dietary consumption of fats on average results in a ratio of n6 to n3 fatty acids of between 7:1 to 15:1 or higher, representing a pro-inflammatory state, lower ratios have been linked to more normal states of inflammation.

Due to the positive effect omega-3 fatty acids are believed to have on arginine levels, and subsequent improvement of muscle synthesis and immune response, and the positive synergy between the two compounds, it is desirable to include omega-3 fatty acids in a composition addressing inflammation that contains arginine.

In addition to arginine deficiency, the diet of the elderly is believed to differ from the younger population in other ways that contribute to inflammation. Specific changes in the intake levels of antioxidants, including but not limited to lutein and other carotenoids and flavanols, by the aged individual compared to young healthy adults reveals an approximate 5 to 7 fold decrease. Similarly, the level of omega-3 fatty acids is believed to decrease from about 3 to 16 times in the elderly. As a result there is a high correlation between low serum levels of these nutrients and elevated inflammatory markers like IL6, IL2 and CRP. Supplementation with strong antioxidants and omega-3 fatty acids may reduce the cytokine response. Further, as shown in FIG. 5, the presences of such antioxidants may act synergistically with omega-3 fatty acids. It is therefore also desirable to include antioxidants such as carotenoids or flavanols in a composition for addressing inflammation.

It may also be desirable to include the amino acid citrulline. It is believed that citrulline can promote T cell proliferation in arginine-deficient media, such that this amino acid may potentially overcome the detrimental effects of Arginase I. T cells are capable of regenerating arginine from citrulline such that citrulline is a useful substitute for arginine in overcoming Arginase I mediated immune suppression. Therefore, it is desirable to include citrulline in a composition for improving immune function and diminishing inflammation, particularly under conditions of arginine deprivation such as a poor, low-protein diet.

Another common problem in the elderly, particularly those fighting infection, is malnutrition or anorexia. In response to infection, serum levels of inflammatory mediators may further be elevated and have a negative effect on the appetite, often causing anorexia. Once older adults get sick, nutritional status is at risk because food intake decreases, particularly of protein and micronutrients. When inadequate nutrient intakes continue for an extended period, a state of undernutrition develops. Undernutrition is a widespread problem among elderly persons receiving formal medical care. In fact, about 23-62% of hospitalized patients, and up to about 85% of nursing home patients, are believed to be undernourished, with up to about 70% of elderly patients suffering from muscle wasting and up to about 60% being hyperinflammed. With inadequate intake of macro and micro nutrients (e.g. up to a 20% decrease in protein and fat along with a shift from unsaturated fat to higher saturated fat intake along with a decrease in the B vitamins, anitoxidants, and others) derived from daily food intake, recommended daily intakes are not achieved, nor are the altered metabolic requirements to support a normal immune system response of the aged individual. As a result a pro-oxidant and hyper-inflamed state exists in the elderly that recommends specialized and targeted nutrient intake in order to improve the healthspan of the individual.

It is believed that an approximate 30% reduction in protein intake by the elderly and corresponding decrease in essential amino acids generates a combined detrimental effect. In the elderly, high protein (meat) products are generally less frequently selected, and high quality protein providing more essential amino acids and arginine are generally specifically reduced in the diet. Similarly, not only is there believed to be an approximate 30% decrease in the amount of dietary fat consumed, but the quality of fat consumed is believed to tend toward high saturated fats. As a result of this shift, the amount of poly-unsaturated fats is believed to be lower by 20%, serving to elevate the ratio of the pro-inflammatory omega-6 fatty acids to the anti-inflammatory omega-3 fatty acids to as high as 15:1. Not surprisingly, these dietary habits are believed to correlate with elevated levels of C-reactive protein (CRP). The average CRP values may be about 2 fold higher than normal levels, and as much as about 5 fold higher in the most nutritionally compromised aged individuals.

These observations on the dietary habits and practices of the elderly suggest that diet alone is not adequate to manage the risk of hyper-inflammation. Because the elderly tend to suffer malnutrition, and because malnutrition (particularly the absence of essential amino acids and arginine, and the presence of high saturated fats) contributes to inflammation, it is desirable that a composition for ameliorating inflammation also provide protein and healthy fats (e.g. omega 3 polyunsaturated fatty acids), and avoid high saturated fats (including omega 6 fatty acids), as well as calories in order to also address and decrease the risk of malnutrition.

A further complication of inflammation in the elderly is an increase in insulin resistance. In vitro studies demonstrate that TNFa, an inflammation mediator, can induce insulin resistance and down-regulate insulin receptor signaling in skeletal muscle. Similarly, other studies have observed an association between chronic wasting in older age with insulin resistance, in parallel with increases in TNFa. This decreased sensitivity of insulin in the aged individual contributes to a decrease in protein synthesis and alters carbohydrate metabolism such that low glycemic index foods are important to the elderly. Recent studies have shown that higher degrees of inflammation are associated with impaired glucose tolerance (IGT) both pre-diabetes and in the case of advanced diabetes. Thus, increasing levels of inflammation are believed to increase the risk of chronic disease and complicate the management of co-morbidities. It is therefore desirable for a dietary treatment of inflammation to be sensitive to potential insulin resistance, particularly if the supplement is also intended to provide calories and energy in order to address malnutrition.

Additional data supports a synergistic effect of nutrients so that the inflammatory response itself can be improved. The nutrient deficiencies of the aging lead to a decreased capacity of the immune-inflammatory cascade and recommend a method that can overcome this deficiency and provide a manner of normal response. Pathways for this normal response are the arginine-arginase pathway as well as in the NOS pathway, both of which are able to modulate inflammation.

Altered caloric intake, an increase in saturated fats, compromised glucose metabolism and decreased activity all contribute to an increase in fatty muscle tissue. This risk increases with age, as the ability to burn/oxidize fat is impaired, putatively in response to mitochondrial dysfunction. Fat tissue induces a kind of low grade inflammatory state. Therefore, with increased caloric intake or higher than normal Body Mass index (BMI), there is a generally less optimal immune status.

### SUMMARY

Because of these and other problems in the alt, disclosed herein are Compositions and methods for managing inflammation in the elderly by delivering a selection of amino acids, including arginine and/or citrulline, in a synergistic ratio with omega-3 fatty acids and carbohydrates with a low glycemic index.

There is described herein, a composition of matter comprising: amino acids, the amino acids further comprising arginine; and omega-3 fatty acids; wherein the amino acids and the omega-3 fatty acids are in a synergistic ratio effective to manage inflammation.

In an embodiment, the composition further comprises a carbohydrate or an antioxidant.

In an embodiment, the amino acids further comprise essential amino acids.

In an embodiment, the composition further comprises an amount of total protein, wherein the arginine comprises between about twenty and about thirty percent of the amount.

In an embodiment, the composition further comprises an amount of total fat, wherein the omega-3 fatty acids comprise between about five and about twenty percent of the amount.

In an embodiment, the composition is in a 200 ml serving, and wherein the omega-3 fatty acids comprise between about 1 and about 2.5 grams per the serving.

In an embodiment the composition further comprising omega-6 fatty acids, wherein the omega-3 fatty acids and the omega-6 fatty acids are in a ratio between about one to two to about one to seven.

In an embodiment, the amino acids further comprise citrulline. The composition may further comprises an amount of total protein, wherein the citrulline comprises between about five and about fifty percent of the amount.

In an embodiment, the composition is a component of means for liquid administration or may comprise an excipient.

There is also described herein, a method of managing inflammation, comprising: having a patient; and delivering to the patient a composition of matter comprising: amino acids, the amino acids further comprising arginine; and omega-3 fatty acids; wherein the amino acids and the omega-3 fatty acids are in a synergistic ratio effective to manage inflammation.

In an embodiment of the method the patient is elderly. The delivering may be performed orally such as by liquid administration.

In an embodiment of the method, the composition further comprises an amount of total protein, and the arginine comprises between about twenty and about thirty percent of the amount.

In another embodiment of the method, the composition further comprises an amount of total fat, and the omega-3 fatty acids comprise between about five and about twenty percent of the amount.

In another embodiment of the method, the composition is in a 200 ml serving, and wherein the omega-3 fatty acids comprise between about 1 and about 2.5 grams per the serving.

In another embodiment of the method the composition further comprises omega-6 fatty acids, wherein the omega-3 fatty acids and the omega-6 fatty acids are in a ratio between about one to two to about one to seven.

In another embodiment of the method the amino acids further comprise citrulline. The composition may further comprise an amount of total protein, and the citrulline comprises between about five and about fifty percent of the amount.

There is also described herein, a method of managing inflammation in the elderly, comprising: generating a composition of matter comprising: amino acids, the amino acids further comprising arginine; and omega-3 fatty acids; wherein the amino acids and the omega-3 fatty acids are in a synergistic ratio effective to manage inflammation; and providing to an elderly patient the composition of matter.

In an embodiment of the method the composition further comprises an amount of total protein, and the arginine comprises between about twenty and about thirty percent of the amount of total protein; and where the composition further comprises an amount of total fat, and the omega-3 fatty acids comprise between about five and about twenty percent of the amount of total fat.

### BRIEF DESCRIPTION OF THE DRAWINGS

FIG. 1 shows a scheme of interaction between mitochondria and aging via pro-inflammatory cytokines.

FIG. 2 shows arginine de novo synthesis during sepsis.

FIG. 3 shows how arginine is believed to stimulate muscle protein synthesis.

FIG. 4 shows an effect of amino acid supplementation on protein synthesis in normal and inflamed and inflamed with DHA conditions.

FIG. 5 shows changes in lutein and DHA and combinations in serum levels following supplementation with an embodiment of the compositions disclosed herein.

FIG. 6 shows pathways of arginine synthesis and breakdown.

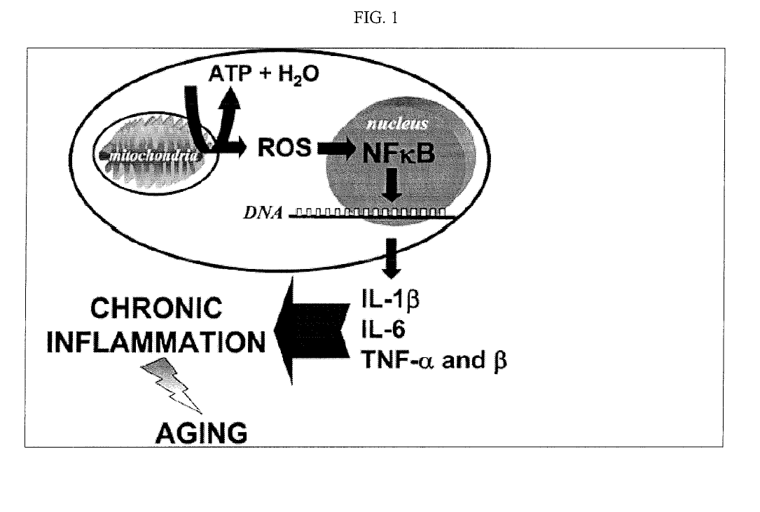

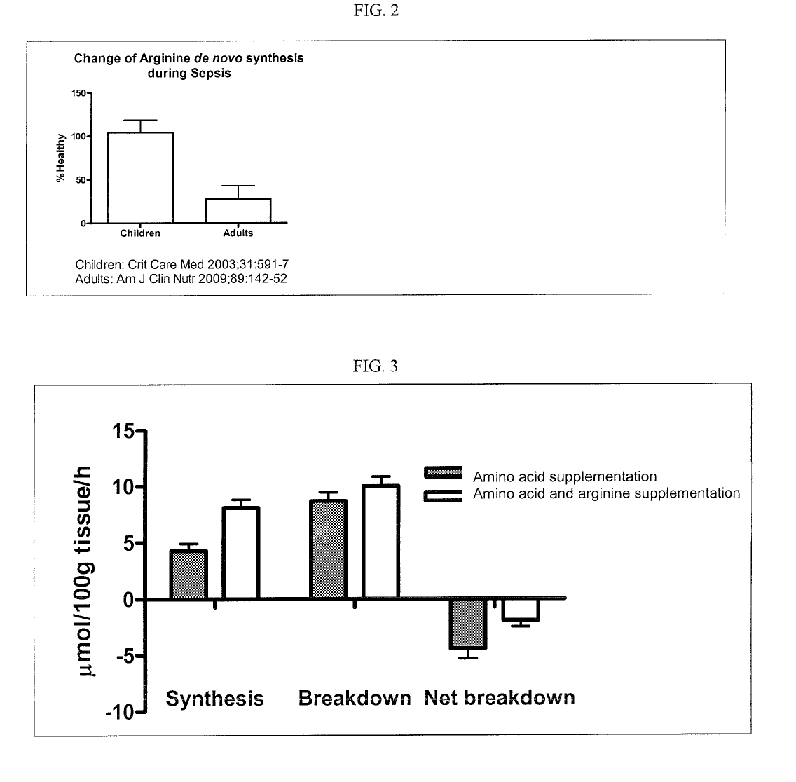

### DESCRIPTION OF PREFERRED EMBODIMENTS

Disclosed herein are compositions and methods for the nutritional control and management of hyper-inflamed conditions in the elderly (including but not limited to individuals over sixty years of age) which may as a result of poor nutritional practices and habits, a weakened immune system, or any other reason. The disclosed compositions and methods provide nutritional compositions, the ingredients of which complimentarily and synergistically reduce and manage the inflammatory response, thereby providing enhanced natural defense mechanisms and protection from infectious challenge. Further, they serve to specifically deliver selected nutrients and supplement the diet of elderly individuals who otherwise would remain in a hyperinflammed condition and more susceptible to compromised health status and infection.

The compositions disclosed herein may more appropriately protect the aged individuals from infections, infectious complications and manage, slow, reduce, or prevent the onset of inflammatory based chronic disease conditions, including muscle wasting, sarcopenia, congestive heart failure, cachexia, diabetes, cognitive decline, COPD, digestive diseases, infections and infectious complications, and acute conditions including the preparation for and recovery from elective surgical procedures. The compositions and methods disclosed herein may slow the aging process and reduce the number of co-morbidities of aging. Additionally, by providing stronger support of inflammatory controls and strengthening the immune response, protection from acute health conditions or procedures in elective surgical care and recovery may be improved.

Generally, described herein are compositions and methods of delivering the compositions wherein the compositions are a synergistic combination comprising arginine and/or citrulline, omega-3 fatty acids, antioxidants, ribose, low glycemic carbohydrates, and compounds for improving the taste or other sensory experiences of delivering the composition.

In an embodiment comprising essential amino acids, the compositions and methods disclosed herein address the reduced ability of the elderly to maintain muscle protein synthesis and thus muscle mass, strength, and function. They may do so by slowing the increased catabolic process. In such an embodiment, protein intake is increased and the composition of amino acids is enhanced with essential amino acids and arginine and citrulline. In an attempt to maintain a more normal rate of protein synthesis in an aged model of synthesis, the composition may comprise a generally higher concentration of arginine and/or citrulline. Total protein with higher levels of selected amino may be achieved by using a blend of amino acids, peptides high in leucine, intact protein sources, and slow release long chain saccharides/carbohydrates in which the proportion of each amino acid and the amount of these carbohydrates may accelerate muscle protein synthesis and muscle protein turnover, and consequently increase muscle mass, strength and physical function.

The amino acids may overcome a depressed ability to stimulate protein synthesis due to inflammation and, by managing or reducing inflammation, achieve more normal levels of muscle protein synthesis. The particular essential amino acids are provided because they cannot be produced in the body and are thus their availability is rate-limiting for protein synthesis. Amino acids may be included in the free form; in combinations of peptides; in combinations of intact protein and free amino acids; in combinations of free amino and peptides; or in combinations of free amino acids, peptides, and proteins.

In an embodiment, the composition may further comprise ingredients that can reduce the level of inflammation, including but not limited to polyunsaturated omega-3 fatty acids, EPA, DHA, or any other functional equivalent. The synergistic combination of such ingredients with arginine and/or citrulline may overcome the negative influence that hyper-inflammation has on muscle energetics and decrease inflammation, oxidative breakdown, and damage to cells in the body, and overcome the negative consequences of inflammation on physiological systems.

In an embodiment, the composition further comprises a low glycemic carbohydrate. These selected carbohydrates may be medium and long chain polysaccharides that are metabolically slow to digest and release low levels of glucose into the blood stream after digestion. The carbohydrate may be any of or a blend of dextrans or multi-dextrans having more than 8 carbon units or chain lengths, malodextrans, sucramaltose, and soluble fibers (such as, but not limited to, NUTRIOSE® (chemicals for use in human and dietetic food), FIBERSOL® (dietary fiber), Sucramalt, or INULIN® (dietary supplement containing inulin)), or any of the functional equivalents. The low glycemic carbohydrate may provide energy needed to produce the new protein without eliciting a significant insulin response. The elderly are generally resistant to the action of insulin, so avoiding an insulin response by using low glycemic carbohydrate will be advantageous to that population.

In addition to or alternatively to a low glycemic carbohydrate or slow release saccharide, the composition may comprise ribose. Ribose may increase the amount of tRNA, which may be useful in supporting protein synthesis when combined with increased availability of the rate-limiting amino acids and arginine provided. Because of the similar functionality of low glycemic carbohydrates, slow release saccharides, and ribose in the compositions and methods disclosed herein, the term “carbohydrate” used herein may refer to all these compounds.

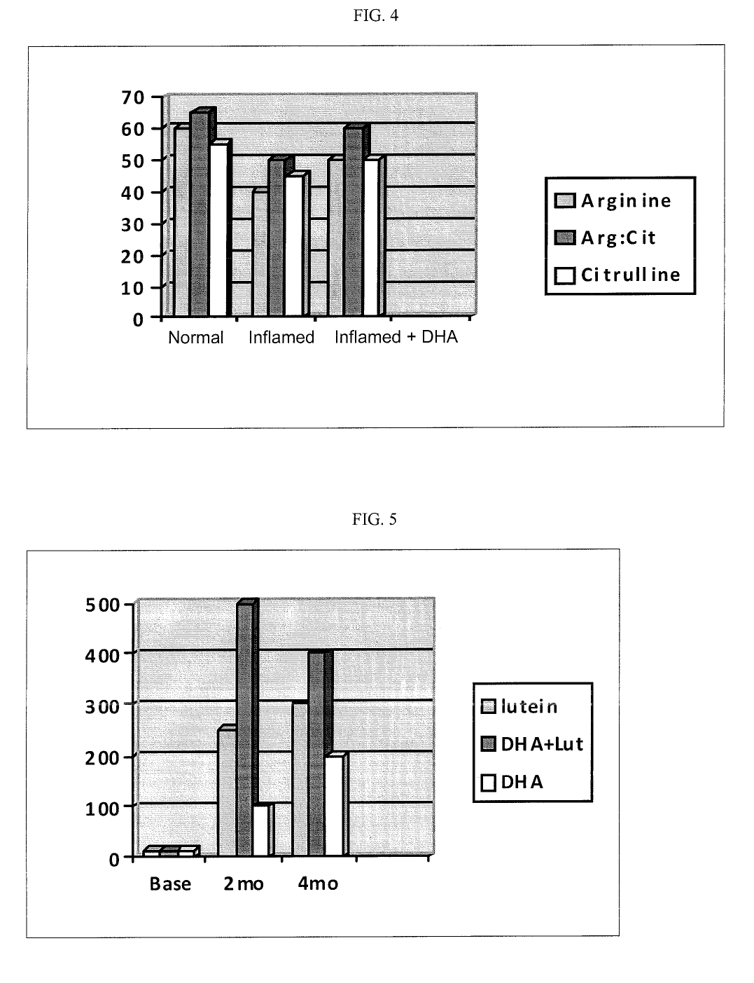

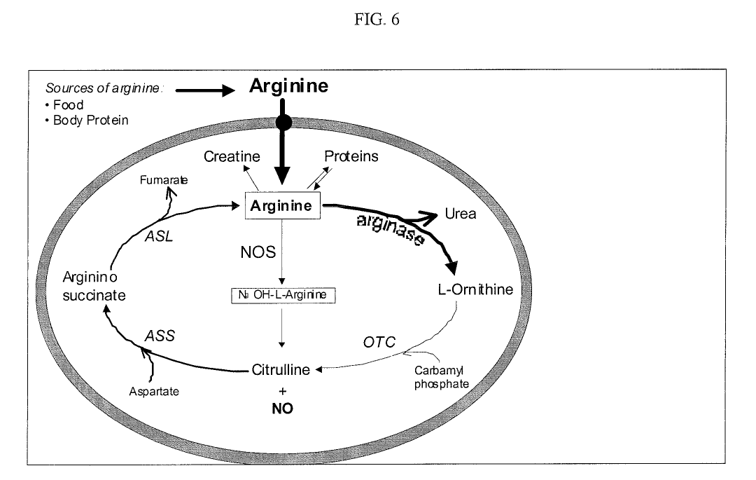

In an embodiment, the composition may further comprise carotenoids, including but not limited to lutein, zeaxanthine, and other functionally equivalent antioxidants and other compounds. As shown in FIG. 5, such compounds may act synergistically with omega-3 fatty acids. Such compounds are often missing from the diet of the elderly.

The disclosed selected active nutrients may be delivered in at any range of ratios and levels of use that, when combined effectively, enhance an immune system response and manage inflammation to protect the individual from infection and help manage chronic diseases affected by inflammation. Preferred ranges of nutrients to naturally control the inflammatory response include a blend with a ratio of citrulline to arginine of 0.25 to 1 to 4 to 1 in a ratio to omega 3 fatty acids of 1 to 0.5 to 3 to 1 where the ratio of omega 3 to omega 6 is greater than 2.5 to 1. Additional ingredients can be included at optional levels of 0.25 g to 2 g for ribose. As a base formula and in conjunction with the ratio of actives described above, essential amino acids should be available at a level of 50% and higher of total protein where the level of leucine is available at a level of between 11 to 25% of total amino acid dry weight.

In an alternative or further embodiment, the composition comprises a protein level where essential amino acids represent a level of between about 50% to 100% of available protein. Alternatively or further, the level of leucine may be between about 11 and 25% of total protein available; arginine may be between about 20 and 30% of total protein available; and citrulline may be between about 5 and 50% of total available protein. The leucine to arginine to citrulline ratio may be between about 0.25:1:0.25 to 1.1:6. In this embodiment, leucine is provided to specifically help reduce muscle wasting.

In an embodiment, the composition comprises a level of omega-3 fatty acids between about 5% to 20% of total available fat (a range of about 1 to 2.5 g per 200 ml serving). The ratio of omega-3 to omega-6 fatty acids may be between about 1:2 to 1:7.

The ratio of carbohydrate may be in the range of about 1:1 to 4:1. In a preferred embodiment, the ratio is about 2:1. In an alternative or further embodiment, the carbohydrate is in a ratio with protein and fat macronutrients at a ratio between about 4:1:1 to 1:1:2. In an alternative or further embodiment, the composition comprises ribose at a level between about 100 mg to 1.0 g.

In an alternative or further embodiment, the composition comprises antioxidants, such as but not limited to carotenoids such as lutein and zeaxanthine, at a level between about 0.25 mg to 30 mg. The level of lutein may be between about 5 to 20 mg. Where the antioxidants are flavanols, in an embodiment the composition comprises flavanols from sources of tea, chocolate, or any other functionally equivalent source at a level of about 100 mg to 3.0 grams of concentrated extract.

Critical aspects of the method of delivery for therapeutic nutrition involve the selection and use of flavors that are not averse to the elderly. In preferred embodiments, flavor selections are low to no bitterness and higher and more robust flavor attributes such as sweetness. These flavor profiles may be achieved by combination effects such as loquot/lime, mango/peach, black cherry/apple, pomegranate/cranberry, plum tea, apple/peach, and blueberry/grape. These flavors may be provided in clear or nearly clear juice liquid forms with no milky residue, achieved by using high acid treatment of whey protein isolates. In an embodiment, the pH may be less than 3.0 and range of 2.0 to 5.0.

In alternative or further embodiments, supplemental minerals may also be included. Suitable minerals may include one or more minerals or mineral sources with a focus on use of critical vitamins or minerals associated with benefit in the aging process. These include vitamin D, calcium, the family of B vitamins, vitamin A, E and C. Non-limiting examples of minerals include, without limitation: chloride, sodium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof. Delivery of such vitamins and minerals in the methods disclosed herein, which may be “active snacking” selected availability of vitamins and minerals should provide not more than 25% of total daily requirements per serving in an ideal range of 15 to 30% of recommended daily intakes.

The compositions may also optionally comprise vitamins. The vitamins may be fat-soluble or water soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. The form of the vitamin may include salts of the vitamin, derivatives of the vitamin, compounds having the same or similar activity of a vitamin, and metabolites of a vitamin.

The composition may also comprise at least one excipient. Non-limiting examples of suitable excipients include a buffering agent, a preservative, a stabilizer, a binder, a compaction agent, a lubricant, a dispersion enhancer, a disintegration agent, a flavoring agent, a sweetener, a coloring agent, and combinations of any of these agents.

In an embodiment, the excipient is a buffering agent. Non-limiting examples of suitable buffering agents include sodium citrate, magnesium carbonate, magnesium bicarbonate, calcium carbonate, and calcium bicarbonate.

The excipient may comprise a preservative. Suitable examples of preservatives include antioxidants, such as alpha-tocopherol or ascorbate, and antimicrobials, such as parabens, chlorobutanol, or phenol.

In an alternative or further embodiment, the excipient may be a binder. Suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof.

In an alternative or further embodiment, the excipient may be a lubricant. Suitable non-limiting examples of lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

The excipient may be a dispersion enhancer. Suitable dispersants may include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In yet another embodiment, the excipient may be a disintegrant. The disintegrant may be a non-effervescent disintegrant. Suitable examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. The disintegrant may be an effervescent disintegrant. Suitable effervescent disintegrants include sodium bicarbonate in combination with citric acid, and sodium bicarbonate in combination with tartaric acid.

In another embodiment, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), sucralose and sodium and calcium salts thereof.

It may be desirable to provide a coloring agent. Suitable color additives include food, drug and cosmetic colors (FD&C), drug and cosmetic colors (D&C), or external drug and cosmetic colors (Ext. D&C). These colors or dyes, along with their corresponding lakes, and certain natural and derived colorants, may be suitable for use in certain embodiments.

The weight fraction of the excipient or combination of excipients in the formulation may be about 30% or less, about 25% or less, about 20% or less, about 15% or less, about 10% or less, about 5% or less, about 2%, or about 1% or less of the total weight of the amino acid composition.

Also contemplated are methods of delivery of the compositions disclosed herein, including but not limited to dosage. The compositions disclosed or made obvious herein may be formulated into a variety of forms and administered by a number of different means. The compositions may be administered orally, rectally, or parenterally, in formulations containing conventionally acceptable carriers, adjuvants, and vehicles as desired. The term “parenteral” as used herein includes subcutaneous, intravenous, intramuscular, or intrasternal injection, or infusion techniques. In an exemplary embodiment, the compounds of the invention are administered orally.

Solid dosage forms for oral administration may include capsules, tablets, caplets, pills, troches, lozenges, powders, and granules. A capsule typically comprises a core material comprising a composition of the invention and a shell wall that encapsulates the core material. The core material may be solid, liquid, or an emulsion. The shell wall material may comprise soft gelatin, hard gelatin, or a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). Some such polymers may also function as taste-masking agents.

Tablets, pills, and the like may be compressed, multiply compressed, multiply layered, and/or coated. The coating may be single or multiple. In one embodiment, the coating material may comprise a polysaccharide or a mixture of saccharides and glycoproteins extracted from a plant, fungus, or microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In another embodiment, the coating material may comprise a protein. Suitable proteins include, but are not limited to, gelatin, casein, collagen, whey proteins, soy proteins, rice protein, and corn proteins. In an alternate embodiment, the coating material may comprise a fat or oil, and in particular, a high temperature melting fat or oil. The fat or oil may be hydrogenated or partially hydrogenated, and preferably is derived from a plant. The fat or oil may comprise glycerides, free fatty acids, fatty acid esters, or a mixture thereof. In still another embodiment, the coating material may comprise an edible wax. Edible waxes may be derived from animals, insects, or plants. Non-limiting examples include beeswax, lanolin, bayberry wax, carnauba wax, and rice bran wax. Tablets and pills may additionally be prepared with enteric coatings.

Alternatively, powders or granules embodying the compositions disclosed and made obvious herein may be incorporated into a food product. The food product may be a drink. Non-limiting examples of a suitable drink include fruit juice, a fruit drink, an artificially flavored drink, an artificially sweetened drink, a carbonated beverage, a sports drink, a liquid diary product, a shake, and so forth.

The compositions may also be in liquid dosage forms for oral administration. Liquid dosage forms include aqueous and nonaqueous solutions, emulsions, suspensions and solutions and/or suspensions reconstituted from non-effervescent granules, containing suitable solvents, preservatives, emulsifying agents, suspending agents, diluents, sweeteners, coloring agents, and flavoring agents. In a preferred embodiment addressing intake and compliance, the volume of delivery of a liquid dosage form may be in a range of about 100 to 180 ml. In a further preferred embodiment, the volume is about 170 ml. Compliance may further improved by specialized bottle design.

The compositions of the invention may be utilized in methods to increase muscle mass, strength and physical function. In an embodiment, the method comprises administering the composition as described above twice per day between meals. The amount per dose may be about 6, 7, 8, 9, 10, 11, 12, 13, 14, 15, 16, 17, 18, 19, 20, 21, 22, 23, 24, 25, 26, 27, 28, 29, or 30 g. Alternatively, the composition may be administered one, two, three, or four times per day, or any number of times that is both feasible and effective.

In an alternative or further embodiment of a method of delivery, the composition may also be used in conjunction with exercise. For example, the composition may given before or immediately after exercise.

While the invention has been disclosed in connection with certain preferred embodiments, this should not be taken as a limitation to all of the provided details. Modifications and variations of the described embodiments may be made without departing from the spirit and scope of the invention, and other, embodiments should be understood to be encompassed in the present disclosure as would be understood by those of ordinary skill in the art.

1. A composition of matter comprising:
amino acids, said amino acids further comprising arginine; and
omega-3 fatty acids;
wherein said amino acids and said omega-3 fatty acids are in a synergistic ratio effective to manage inflammation.

2. The composition of claim 1 further comprising a carbohydrate.

3. The composition of claim 1 further comprising an antioxidant.

4. The composition of claim 1 wherein said amino acids further comprise essential amino acids.

5. The composition of claim 1 further comprising an amount of total protein, wherein said arginine comprises between about twenty and about thirty percent of said amount.

6. The composition of claim 1 further comprising an amount of total fat, wherein said omega-3 fatty acids comprise between about five and about twenty percent of said amount.

7. The composition of claim 1 wherein said composition is in a 200 ml serving, and wherein said omega-3 fatty acids comprise between about 1 and about 2.5 grams per said serving.

8. The composition of claim 1 further comprising omega-6 fatty acids, wherein said omega-3 fatty acids and said omega-6 fatty acids are in a ratio between about one to two to about one to seven.

9. The composition of claim 1 wherein said amino acids further comprise citrulline.

10. The composition of claim 9 further comprising an amount of total protein, wherein said citrulline comprises between about five and about fifty percent of said amount.

11. The composition of claim 1 wherein said composition is a component of means for liquid administration.

12. The composition of claim 1 further comprising an excipient.

13. A method of managing inflammation, comprising:
having a patient; and
delivering to said patient a composition of matter comprising:
amino acids, said amino acids further comprising arginine; and
omega-3 fatty acids;
wherein said amino acids and said omega-3 fatty acids are in a synergistic ratio effective to manage inflammation.

14. (canceled)

15. The method of claim 13 wherein said patient is elderly.

16. The method of claim 13 wherein said delivering is oral.

17. The method of claim 16 wherein said delivering is by liquid administration.

18. The method of claim 13 wherein said composition further comprises an amount of total protein, and said arginine comprises between about twenty and about thirty percent of said amount.

19. The method of claim 13 wherein said composition further comprises an amount of total fat, and said omega-3 fatty acids comprise between about five and about twenty percent of said amount.

20. The method of claim 13 wherein said composition is in a 200 ml serving, and wherein said omega-3 fatty acids comprise between about 1 and about 2.5 grams per said serving.

21. The method of claim 13 wherein said composition further comprises omega-6 fatty acids, wherein said omega-3 fatty acids and said omega-6 fatty acids are in a ratio between about one to two to about one to seven.

22. The method of claim 13 wherein said amino acids further comprise citrulline.

23. The method of claim 22 wherein said composition further comprises an amount of total protein, and said citrulline comprises between about five and about fifty percent of said amount.

24. A method of managing inflammation in the elderly, comprising:
generating a composition of matter comprising:
amino acids, said amino acids further comprising arginine; and
omega-3 fatty acids;
wherein said amino acids and said omega-3 fatty acids are in a synergistic ratio effective to manage inflammation; and
providing to an elderly patient said composition of matter.

25. The method of claim 24 wherein said composition further comprises an amount of total protein, and said arginine comprises between about twenty and about thirty percent of said amount of total protein; and where said composition further comprises an amount of total fat, and said omega-3 fatty acids comprise between about five and about twenty percent of said amount of total fat.
